(19) United States
(12) Patent Application Publication
(10) Pub. No.: US 2024/0123105 A1
(43) Pub. Date: Apr. 18, 2024

(54) SHOPPING CART HANDLE SANITIZING SYSTEM

(71) Applicant: Marco Maldonado, Hebbronville, TX (US)

(72) Inventor: Marco Maldonado, Hebbronville, TX (US)

(21) Appl. No.: 17/965,015

(22) Filed: Oct. 13, 2022

(51) Int. Cl. A61L 2/26; A61L 2/18; B62B 3/14; B65D 25/22; B65D 43/16

(52) U.S. Cl. CPC A61L 2/26; A61L 2/18; B62B 3/14; B65D 25/22; B65D 43/16

(57) ABSTRACT

A shopping cart handle sanitizing system for containing antibacterial gel for sanitizing includes a shopping cart. The shopping cart includes a frame, a basket, a handle, and a plurality of wheels. A container has a lid positioned thereon for closing the container. A coupler is attached to the container to releasably secure the container to the shopping cart. An antibacterial gel is positioned in the container which is positionable on the handle to sanitize the handle.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to shopping cart attachment devices and more particularly pertains to a new shopping cart attachment device for containing antibacterial gel for sanitizing a handle of the shopping cart.

### (2) Description of Related Art Including Information Disclosed Under 37 CFR 1.97 and 1.98

The prior art relates to shopping cart cleaning assemblies and systems used for sanitizing a shopping cart before usage.

### (h) BRIEF SUMMARY OF THE INVENTION

An embodiment of the disclosure meets the needs presented above by generally comprising a shopping cart including a frame, a basket mounted on the frame, a child seat, an elongated handle attached to the frame, and a plurality of wheels being rotatably coupled to the frame. A container has a lid positioned thereon for closing the container. A coupler is attached to the container and is releasably secures the container to the shopping cart. An antibacterial gel is positioned in the container and the antibacterial gel is positionable on the handle to sanitize the handle.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front isometric view of a shopping cart handle sanitizing system according to an embodiment of the disclosure.

FIG. 2 is a rear isometric view of an embodiment of the disclosure.

FIG. 3 is a cross-sectional view of an embodiment of the disclosure taken from FIG. 1 of Line 3-3.

FIG. 4 is a front isometric view of an embodiment of the disclosure.

FIG. 5 is a front view of an embodiment of the disclosure.

FIG. 6 is an in-use view of an embodiment of the disclosure.

FIG. 7 is an in-use view of an embodiment of the disclosure.

### (j) DETAILED DESCRIPTION OF THE INVENTION

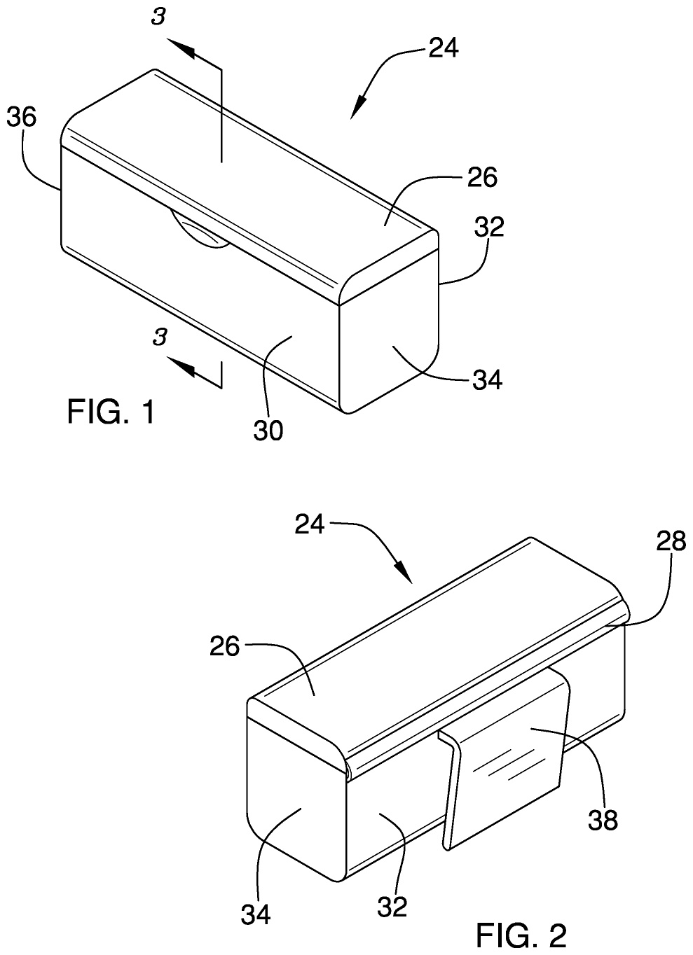

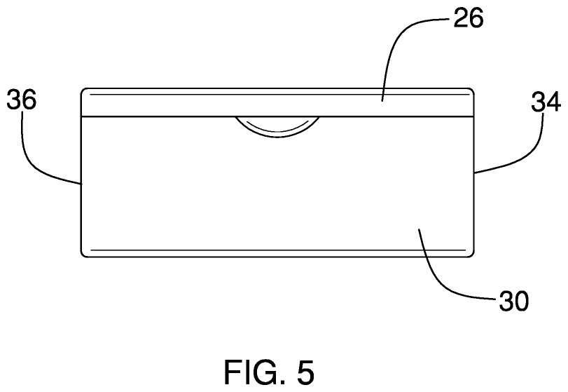

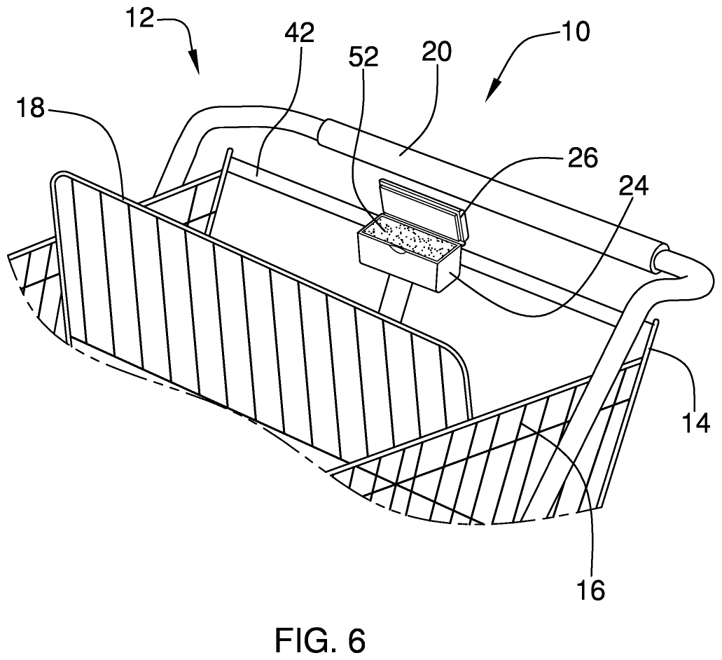

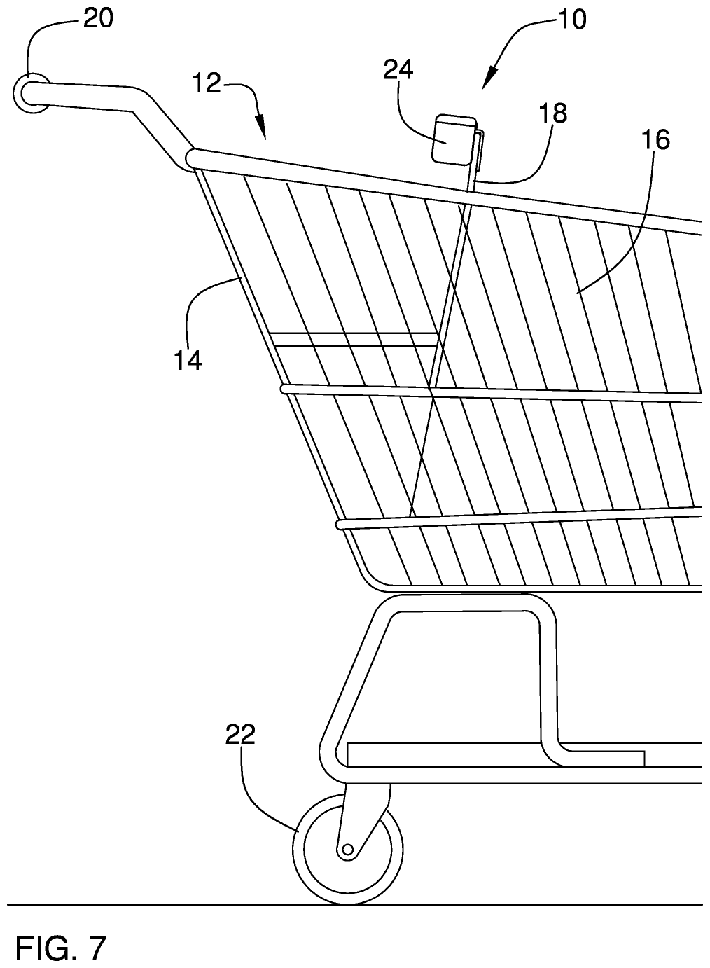

With reference now to the drawings, and in particular to FIGS. 1 through 7 thereof, a new shopping cart attachment device embodying the principles and concepts of an embodiment of the disclosure and generally designated by the reference numeral 10 will be described.

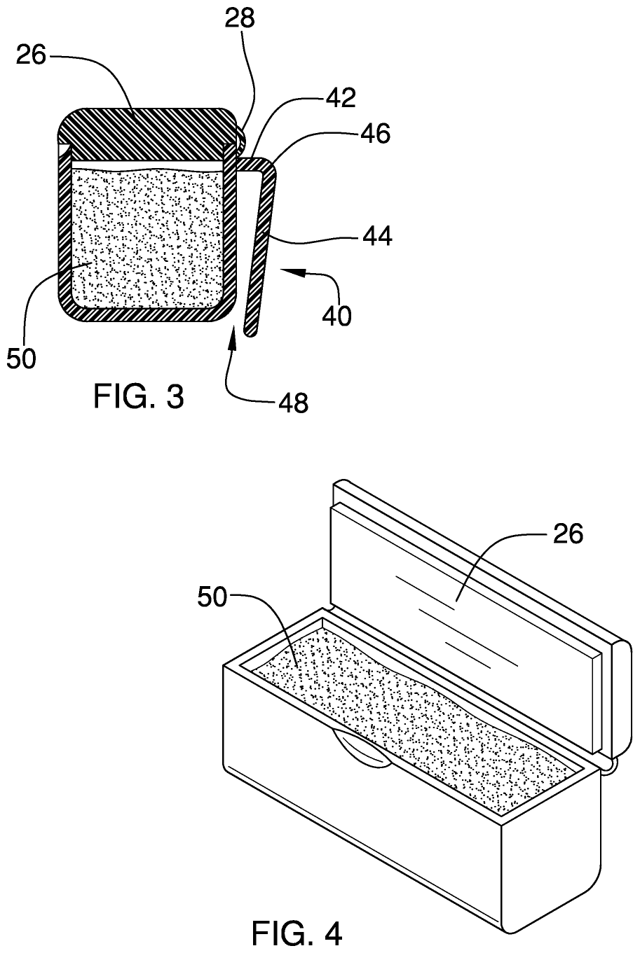

As best illustrated in FIGS. 1 through 7, the shopping cart handle sanitizing system 10 generally comprises a shopping cart 12 including a frame 14, a basket 16 mounted on the frame 14 including a child seat 18, a handle 20 attached to the frame 14 and which is elongated, and a plurality of wheels 22 being rotatably coupled to the frame 14. A container 24 has a lid 26 positioned thereon for closing the container 24 wherein the lid 26 is pivotally coupled to the container 24. This may be accomplished, for example, by a living hinge 28, though a conventional hinge may be employed. The container 24 has a front side 30 and a back side 32 and the lid 26 is coupled to the back side 32 of the container 24. The container 24 has a length less than 4 inches from the first lateral wall 34 to a second lateral 36 and a height and depth each being less than 3.0 inches. The lid 26 may be retained in a closed position by a friction fit lid wherein a notch in the container may be used to engage the lid 26 with a person's finger. As can be seen in FIG. 3, the lid may include a lower portion extending into the container to frictionally engage a wall of the container.

A coupler 38 is attached to the container 24 and is configured to releasably secure the container 24 to the shopping cart 12. The coupler 38 is attached to the back side 32 of the container 24 and may comprise a clip 40 configured to receive an upper edge 42 of the basket 16 such that the back side 32 abuts the basket 16. The clip 40 includes a base 44 and an arm 46. The base 44 of the clip 40 is positioned adjacent to the lid 26. The base 44 has an end 48 and the arm 46 protrudes downwardly from the end 48. The arm 46 is spaced from the back side 32 of the container 24 and defines an access space 50 where the shopping cart 12 may be inserted into the clip 40. The arm 46 may be biased toward the container 24 to frictionally engage the basket 16 or other areas of the shopping cart 12. A conventional antibacterial gel 52 is positioned in the container 24. The antibacterial gel 52 is positionable on the handle 20 with either a person's hand, cloth, tissue or the like.

In use, the coupler 38 of the container 24 secures to the shopping cart 12 by inserting the upper edge 42 of the basket 16 into the clip 40. The lid 26 is pivotally opened to provide access to the antibacterial gel 52. The antibacterial gel 52 is removable from the container 24 and may be applied to the handle 20 of the shopping cart 12 to sanitize the handle 20 of the shopping cart 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A handle sanitizing system comprising:
a shopping cart including:
a frame;
a basket mounted on the frame, said basket including a child seat;
a handle attached to the frame, said handle being elongated;
a plurality of wheels being rotatably coupled to said frame;
a container having a lid positioned thereon for closing said container;
a coupler being attached to said container and being configured to releasably secure said container to said shopping cart; and
an antibacterial gel being positioned in said container, wherein the antibacterial gel is positionable on the handle to sanitize the handle.

2. The handle sanitizing system of claim 1, further comprising said lid pivotally coupled to said container.

3. The handle sanitizing system of claim 2, further comprising said container having a front side and a back side.

4. The handle sanitizing system of claim 3, further comprising said lid being coupled to said back side of said container.

5. The handle sanitizing system of claim 1, further comprising said container having a length less than 4 inches from said first lateral wall to a second lateral.

6. The handle sanitizing system of claim 5, further comprising said container having a height and depth each being less than 3.0 inches.

7. The handle sanitizing system of claim 3, further comprising said coupler being attached to said back side said container.

8. The handle sanitizing system of claim 1, further comprising said coupler comprising a clip configured to receive an upper edge of the basket such that the back side abuts the basket.

9. A handle sanitizing system comprising:
a shopping cart including:
a frame;
a basket mounted on the frame, said basket including a child seat;
a handle attached to the frame, said handle being elongated;
a plurality of wheels being rotatably coupled to said frame;
a container having a lid positioned thereon for closing said container, said lid pivotally coupled to said container, said container having a front side and a back side, said lid being coupled to said back side of said container, said container having a length less than 4 inches from said first lateral wall to a second lateral, said housing having a height and depth each being less than 3.0 inches;
a coupler being attached to said container and being configured to releasably secure said container to said shopping cart, said coupler being attached to said back side said container, said coupler comprising a clip configured to receive an upper edge of the basket such that the back side abuts the basket; and
an antibacterial gel being positioned in said container, wherein the antibacterial gel is positionable on the handle to sanitize the handle.
